(19) United States
(12) Patent Application Publication
(10) Pub. No.: US 2010/0062969 A1
(43) Pub. Date: Mar. 11, 2010

(54) HYDROPHILIC POLYMER-CONJUGATED LIPIDS FOR PEPTIDE AND PROTEIN FOLDING DISORDERS

(76) Inventors: Hayat Onyuksel, Western Spring, IL (US); Israel Rubinstein, Highland Park, IL (US)

Correspondence Address: POLSINELLI SHUGHART PC, 700 W. 47TH STREET, SUITE 1000, KANSAS CITY, MO, 64112-1802, US

(21) Appl. No.: 12/296,238

(22) PCT Filed: Apr. 6, 2007
(86) PCT No.: PCT/US07/08660

§ 371 Date: Nov. 12, 2009

Related U.S. Application Data
(60) Provisional application No. 60/790,297, filed on Apr. 7, 2006.

(51) Int. Cl. A61K 38/00; A61K 31/685

(52) U.S. Cl. 514/2; 514/76

(57) ABSTRACT

The present invention provides a method of correcting peptide or protein misfolding, which can be used to treat peptide and protein disorder in a mammalian subject. The method comprises administering to the mammalian subject, preferably a human subject, an effective amount of a composition comprising sterically stabilized simple micelles (SSM) of a hydrophilic polymer-conjugated lipid or sterically stabilized mixed micelles (SSMM) of a hydrophilic polymer-conjugated lipid and a water-insoluble lipid. The composition may further comprise a biologically active compound, such as but not limited to vasoactive intestinal peptide (VIP), associated with the SSM or SSMM.

### CROSS-REFERENCE TO RELATED APPLICATION

This application claims the priority of Unites States provisional application Ser. No. 60/790,297 filed Apr. 7, 2006, which is incorporated herein by reference and made a part hereof.

### FEDERALLY SPONSORED RESEARCH OR DEVELOPMENT

This invention was made with United States government support under National Institutes of Health grant numbers AG024026; HL072323; RR015482; and Army Medical Research and Material Command DAMD17-02-1-0415. The United States government has certain rights in this invention.

### BACKGROUND OF THE INVENTION

1. Field of the Invention

The present invention is related generally to compositions of sterically stabilized simple micelles (SSM) of a hydrophilic polymer-conjugated lipid or sterically stabilized mixed micelles (SSMM) of a hydrophilic polymer-conjugated lipid and a water-insoluble lipid, and their use for correcting peptide and protein misfolding, which can be used to treat peptide and protein folding disorders.

2. Background of the Invention

The present invention is related generally to compositions of sterically stabilized simple micelles (SSM) of a hydrophilic polymer-conjugated lipids or sterically stabilized mixed micelles (SSMM) of a hydrophilic polymer-conjugated lipid and a water-insoluble lipid, and their use for correcting peptide and protein misfolding, which can be used to treat peptide and protein folding disorders. It is estimated there are perhaps 100,000 different types of proteins in the human body which carry out various vital biological functions (Dobson, C. M., Principles of protein folding, misfolding and aggregation: Seminars in Cell &Dev. Bio. 2004; 15:3-16). Each protein must fold into its correct three-dimensional conformation to achieve its biological function. Protein misfolding and aggregation are known to contribute to many diseases such as alpha-1 antitrypsin deficiency, cystic fibrosis, diabetes type II, hemolytic anemia, Alzheimer's disease for claims because we have data in examples, transmissible spongiform encephalopathies, serpin-deficiency disorders, Huntington disease, Amyotrophic Lateral Sclerosis, Parkinson disease, spinocerebellar ataxias, dialysis-related amyloidosis, polyglutamine diseases, Down's syndrome, Fabry, other gangliosidosis and cataract.

One of the applications for the present invention is the treatment of Alzheimer's Disease (AD). AD is the most common form of dementia afflicting the elderly population; more so in developed countries with higher life expectancy ratios and has tremendous impact on the community. This problem has intensified more than ever in the United States due to aging of the baby boomer generation. Although several treatment modalities are in existence, they are limited by their symptomatic nature and are based on neurotransmitter replenishment strategies. A gradual paradigm shift in research is occurring from symptomatic therapy to mechanism based approaches where the targets are the pathophysiological hallmarks of AD such as plaque formation, neuroinflammation and taupathy.

AD is due to the aberrant aggregation of β-amyloid (Aβ). Aβ is a hydrophobic peptide responsible for the development of extracellular neuritic plaques in the brain which are a classical hallmark of AD. Biochemical and genetic reports have implicated these plaques in the pathophysiological process of AD (Selkoe D. Alzheimer's disease: genes, proteins, and therapy. Physiol Rev 2001; 81(2):741-766). A key component of the senile neuritic plaque is a central core containing variants of a 38-43 amino acid peptide commonly referred to as β-amyloid (Aβ) due to its high pre-disposition to form β-sheets (Masters C, Simms G, Weinman N, Multhaup G, McDonald B, Beyreuther K. Amyloid plaque core protein in Alzheimer disease and Down syndrome. Proc Natl Acad Sci USA. 1985; 82(12):4245-4249). Altered proteolytic processing and sequential cleavage of transmembrane amyloid precursor protein (APP) by secretase enzymes result in formation of small Aβ fragments (˜4 kDa) of different lengths, primarily 40 (Aβ-40) and 42 (Aβ-42) residues. These fragments agglomerate to form a cascade of intermediate species (including oligomers and protofibrils) which finally culminate in the development of neurotoxic amorphous β-sheeted fibrillar aggregates (Haass C, Selkoe D. Alzheimer's disease. A technical KO of amyloid-beta peptide. Nature 1998; 391(6665):339-340; Lorenzo A, Yankner B. Beta-amyloid neurotoxicity requires fibril formation and is inhibited by congo red. Proc Natl Acad Sci USA 1994; 91(25):12243-12247; Serpell L. Alzheimer's amyloid fibrils: structure and assembly. Biochim Biophys Acta 2000; 1502(1):16-30). Although development and progression of AD is characterized by multiple pathogenic events that include neurofibrillary tangles, neuroinflammation and genetic mutations (Selkoe D. Alzheimer's disease: genes, proteins, and therapy. Physiol Rev 2001; 81(2):741-766; Smith M, Drew K, Nunomura A, Takeda A, Hirai K, Zhu X, Atwood C, Raina A, Rottkamp C, Sayre L, Friedland R, Perry G. Amyloid-beta, tau alterations and mitochondrial dysfunction in Alzheimer disease: the chickens or the eggs? Neurochem Int 2002; 40(6):527-531), there is compelling evidence implicating Aβ-42 aggregation as a pivotal player in the etiology of AD (Hardy J, Selkoe D. The amyloid hypothesis of Alzheimer's disease: progress and problems on the road to therapeutics. Science 2002; 297(5580):353-356). This canonical view of attributing Aβ as the key player in AD etiology, often referred to as the Amyloid Hypothesis, has received almost unanimous acceptance over the last two decades. Several researchers advocate mechanism based therapeutic approaches that target the amyloid cascade through inhibition and clearance of Aβ aggregates. Along these lines, Tramiprosate (ALZHEMED™), an Aβ fibrillogenesis inhibitor has entered Phase III clinical testing in US and Canada (Geerts H. NC-531 (Neurochem). Curr Opin Investig Drugs. 2004 January; 5(1):95-100). We believe that AD progression can be slowed down significantly if aggregation and transformation of Aβ from a monomeric soluble α-helical form to an insoluble amyloidogenic β-sheeted conformation is inhibited.

When located as an element of APP in the transmembrane region of the cell bilayer, Aβ exhibits non-amyloidogenic α-helical conformation (Schroeder F, Jefferson J, Kier A, Knittel J, Scallen T, Wood W, Hapala I. Membrane cholesterol dynamics: cholesterol domains and kinetic pools. Proc Soc Exp Biol Med 1991; 196(3):235-252). Aβ aggregation, in part, can be attributed to the loss of this structural context (provided by cell bilayer) on secretase mediated APP cleavage. To this effect, it has been observed that Aβ-42 also exhibits a significant amount of α-helical character in membrane mimicking environments (Kohno T, Kobayashi K, Maeda T, Sato K, Takashima A. Three-dimensional structures of the amyloid beta peptide (25-35) in membrane-mimicking environment. Biochemistry 1996; 35(50):16094-16104). For example, it has been shown that several hydrophobic proteins and peptides penetrate into the hydrophobic core of sodium dodecyl sulfate (SDS) micelles and adopt α-helical conformation (Pervushin K, Orekhov V, Popov A, Musina L, Arseniev A. Three-dimensional structure of (1-71) bacterioopsin solubilized in methanol/chloroform and SDS micelles determined by 15N-1H heteronuclear NMR spectroscopy. Eur J Biochem 1994; 219(1-2):571-583; Rizo J, Blanco F, Kobe B, Bruch M, Gierasch L. Conformational behavior of Escherichia coli OmpA signal peptides in membrane mimetic environments. Biochemistry 1993; 32(18):4881-4894; Waterhous D, Johnson W, Jr. Importance of environment in determining secondary structure in proteins. Biochemistry 1994; 33(8):2121-2128). However, therapeutic utilization of such membrane mimicking surfactants is greatly limited by their relatively high critical micelle concentration (CMC) and undue toxicity. We have previously demonstrated that several amphiphilic peptides associate with biocompatible and biodegradable nanosized PEGylated phospholipid micelles and change their conformation to α-helix resulting in increased stability and bioactivity (Gandhi S, Tsueshita T, Onyuksel H, Chandiwala R, Rubinstein I. Interactions of human secretin with sterically stabilized phospholipid micelles amplify peptide-induced vasodilation in vivo. Peptides 2002; 23(8):1433-1439; Tsueshita T, Gandhi S, Onyuksel H, Rubinstein I. Phospholipids modulate the biophysical properties and vasoactivity of PACAP-(1-38). J Appl Physiol 2002; 93(4):1377-1383). PEGylated phospholipid micelles provide a hydrophobic milieu amenable to confine Aβ-42 in non amyloidogenic α-helix conformation thereby attenuating its aggregation potential.

Sterically stabilized simple micelles (SSM) are formed spontaneously and reproducibly in aqueous environments when a hydrophilic polymer such as polyethylene glycol (PEG) grafted diacyl lipids are present at super critical micelle concentrations. Steric stabilization refers to the attachment of hydrophilic polymer to phospholipid head groups which renders the micelle “stealth” by providing a physico-mechanical barrier and preventing complement opsonization and liver sequestration (Onyuksel H, Ikezaki H, Patel M, Gao X P, Rubinstein I. A novel formulation of VIP in sterically stabilized micelles amplifies vasodilation in vivo. Pharm Res 1999; 16(1):155-160). SSM overcome the limitations of conventional detergent micelles due to their much lower CMC (μM vs. mM range), hence offering an attractive safety profile (Ashok B, Arleth L, Hjelm R P, Rubinstein I, Onyuksel H. In vitro characterization of PEGylated phospholipid micelles for improved drug solubilization: effects of PEG chain length and PC incorporation. J Pharm Sci 2004; 93(10):2476-2487; Onyuksel H, Ikezaki H, Patel M, Gao X P, Rubinstein I. A novel formulation of VIP in sterically stabilized micelles amplifies vasodilation in vivo. Pharm Res 1999; 16(1):155-160). DSPE-PEG₂₀₀₀ (1,2-Distearoyl-sn-glycero-3-phosphoethanolamine-N-methoxy-poly(ethylene glycol 2000) that we used as an example in the present disclosure is already approved for use in humans by the FDA, albeit for different indications.

The solubilization potential of SSM can further be improved by including a water insoluble lipid such as phosphatidylcholine (PC) to form sterically stabilized mixed micelles (SSMM). Size and solubilization potential of SSMM vary with chain length of the polymer and the content of the water insoluble lipid (Krishnadas A, Rubinstein I, Onyuksel H. Sterically stabilized phospholipid mixed micelles: in vitro evaluation as a novel carrier for water-insoluble drugs. Pharm Res 2003; 20:297-302; Ashok B, Arleth L, Hjelm R P, Rubinstein I, Onyuksel H. In vitro characterization of PEGylated phospholipid micelles for improved drug solubilization: effects of PEG chain length and PC incorporation. J Pharm Sci 2004; 93:2476-87).

The present invention demonstrates the biophysical effect of biocompatible nanosized sterically stabilized micelles (SSM) comprising hydrophilic polymer-conjugated phospholipids on the secondary structure of proteins. Examples are provided for using nanosized (˜14 nm) PEGylated phospholipid micelles on the secondary structure of Aβ-42, its aggregation behavior and neurotoxicity and their potential use as a therapeutic aid for intervention in the Amyloid Cascade. We chose to study Aβ-42 fragment amongst several other variants since biochemical analysis of the amyloid plaque demonstrated that Aβ-42 aggregated more rapidly (Roher A, Lowenson J, Clarke S, Woods A, Cotter R, Gowing E, Ball M. beta-Amyloid-(1-42) is a major component of cerebrovascular amyloid deposits: implications for the pathology of Alzheimer disease. Proc Natl Acad Sci USA 1993; 90(22):10836-10840) and was responsible for seeding and aggregation of other Aβ species in the amyloid core (Jarrett J, Berger E, Lansbury P, Jr. The C-terminus of the beta protein is critical in amyloidogenesis. Ann N Y Acad Sci 1993; 695:144-148).

These and other aspects and attributes of the present invention will be discussed with reference to the following drawings and accompanying specification.

### BRIEF SUMMARY OF THE INVENTION

In an embodiment, the present invention provides a method for treating a peptide and protein folding disorder in a mammalian subject, preferably a human subject, by administering an effective amount of a composition comprising sterically stabilized simple micelles (SSM) of a hydrophilic polymer-conjugated lipid or sterically stabilized mixed micelles (SSMM) of a hydrophilic polymer-conjugated lipid and a water-insoluble lipid to the subject. The hydrophilic polymer-conjugated lipid is preferably a phospholipid such as distearoyl phosphatidylethanolamine. A preferred hydrophilic polymer is polyethylene glycol (PEG) at molecular weight of from about 1000 to about 5000. In another embodiment, the hydrophilic polymer-conjugated lipid is distearoyl phosphatidylethanolamine polyethylene glycol 2000 (DSPE-PEG₂₀₀₀). Preferably, the water-insoluble lipid is phosphatidylcholine. Examples of the peptide and protein folding disorder include, but not limited to, alpha-1 antitrypsin deficiency, cystic fibrosis, diabetes type II, hemolytic anemia, Alzheimer's disease for claims because we have data in examples, transmissible spongiform encephalopathies, serpin-deficiency disorders, Huntington disease, Amyotrophic Lateral Sclerosis, Parkinson disease, spinocerebellar ataxias, dialysis-related amyloidosis, polyglutamine diseases, Down's syndrome, Fabry, other gangliosidosis and cataract. Optionally, the SSM may further comprise a biologically active compound associated with SSM or SSMM. The biologically active compound is preferably an amphaphtic peptide such as, but not limited to, vasoactive intestinal peptide (VIP), growth hormone releasing factor (GRF), peptide histidine isoleucine (PHI), peptide histidine methionine (PHM), pituitary adenylate cyclase activating peptide (PACAP), gastric inhibitory hormone (GIP), hemodermin, the growth hormone releasing hormone (GHRH), sauvagine and urotensin I, secretin, glucagon, galanin, endothelin, calcitonin, α₁-proteinase inhibitor, angiotensin II, corticotropin releasing factor, antibacterial peptides and proteins in general, surfactant peptides and proteins, α-MSH, adrenolmedullin, ANF, IGF-1, α2 amylin, orphanin, or orexin. The composition of the present invention can be delivered by a route such as, but not limited to, intranasally, intravenously, intra-ventrcularly, intracisternally, subcutaneously, topically, intra-thecally, rectally, vaginally, trans-cutaneously, inhalation, sub-lingually, intra-ocular, ocular or orally.

The present invention further provides a method for treating Alzheimer's Disease (AD) in a mammalian subject by administering to the subject a composition comprising sterically stabilized simple micelles (SSM) of a hydrophilic polymer-conjugated lipid or sterically stabilized mixed micelles (SSMM) of a hydrophilic polymer-conjugated lipid and a water-insoluble lipid. The subject is preferably a human subject. In a preferred embodiment, the hydrophilic polymer-conjugated lipid is distearoyl phosphatidylethanolamine polyethylene glycol 2000 (DSPE-PEG₂₀₀₀). A preferred water-insoluble lipid is phosphatidylcholine. The composition may further comprise a biologically active compound suitable for treating AD. A preferred biologically active compound is from the glucagon/sercretin family of peptides such as, but not limited to, vasoactive intestinal peptide (VIP) and pituitary adenylate cyclase activating peptide (PACAP) wherein the PACAP is a L-isomer or D-isomer. In yet a preferred embodiment, the composition is administered intranasally.

The present invention still further provides a method for treating Alzheimer's Disease (AD) in a mammalian subject by administering to the subject an effective amount of a biologically active compound of a member of glucagon/secretin family of peptides, such as, but not limited to vasoactive intestinal peptide (VIP) and pituitary adenylate cyclase activating peptide (PACAP) wherein the PACAP is a L-isomer or D-isomer associated with sterically stabilized simple micelles (SSM) of a hydrophilic polymer-conjugated lipid or sterically stabilized mixed micelles (SSMM) of a hydrophilic polymer-conjugated lipid and a water-insoluble lipid. The subject is preferably a human subject. In a preferred embodiment, the hydrophilic polymer-conjugated lipid is distearoyl phosphatidylethanolamine polyethylene glycol 2000 (DSPE-PEG₂₀₀₀). In yet another preferred embodiment, the water-insoluble lipid is phosphatidylcholine. The composition is preferably administered intranasally.

### BRIEF DESCRIPTION OF THE DRAWINGS

FIG. 1 shows the effect of PEGylated lipids on Aβ-42 aggregation by turbidimetry assay and determination of optimal peptide:lipid ratio. An increase in OD is directly correlated to aggregation. Data represents mean OD of 3 independent experiments (n=3, * p<0.05 compared to Aβ-42 in buffer). Error bars represent standard deviation (S.D.);

FIG. 2 shows the effect of PEGylated lipid on Aβ-42 aggregation by Congo red assay. Data represent the mean OD of 3 independent experiments (* p<0.05 compared to Aβ-42 in buffer). Error bars represent standard deviation;

FIG. 3 shows the effect of PEGylated lipid on Aβ-42 aggregation by fluorometric thioflavine-T assay. Increase in relative fluorescence units (RFU) is proportional to fibril formation. (n=3, * p<0.05 compared to Aβ-42 in buffer). Error bars represent standard deviation;

FIG. 4 is a representative size analysis by quasi-elastic light scattering. (A) Aβ-42 in buffer: After 2 h of incubation, bimodal heterogeneous distribution is observed. 88% of the particles have average diameter of 36.7 nm (±6.2 nm), 12% of the particles have an average size of 134.4 nm (±31.2); (B) Aβ-42 in SSM: After 2 h of incubation, 100% of the particles form a single peak with 11.2±2.3 nm;

FIG. 5 is a representative Electron micrographs of (A) Aβ-42 in buffer (B) PEGylated lipid associated Aβ-42 (c) SSM;

FIG. 6 shows the effect of PEGylated lipids on Aβ-42 induced cytotoxicity. A significant reduction in Aβ-42 induced cytotoxicity is observed in cells treated with PEGylated lipid associated Aβ-42. (n=3, * p<0.05 compared respective §). Error bars represent standard deviation;

FIG. 7 is a schematic presentation of proposed mechanisms for Aβ-42 interaction with PEGylated lipid micelles and its monomers. PEGylated phospholipid micelles provide a hydrophobic environment to preserve Aβ-42 in α-helical conformation; thereby preventing its transformation to pathogenic β-sheeted aggregates (k₁ is significantly reduced). PEGylated lipid monomers coat the high energy domains (“hot-spots”) on the initial aggregates and avert their further interaction and aggregation (k₃ is significantly reduced);

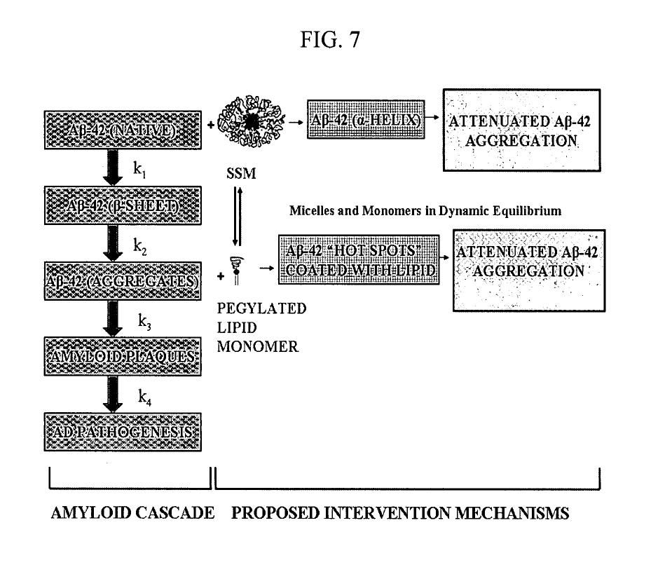

FIG. 8 shows images of gross dissected brain (A) Dorsal part under room light (B) dorsal part under hand held UV lamp showing fluorescence signal (C) fluorescent intensity measurements of mice brain tissue homogenates treated with SSM-QD intranasally or via direct brain injection;

FIG. 9A is a profile of % intact and degraded native VIP and FIG. 9B is a profile of % of intact VIP associated with SSM. Each time point is N=4 samples, data is mean±SEM, * p<0.05;

FIG. 10 are the Lipid:VIP saturation curves in SSM and SSMM determined using fluorescent spectroscopy. Ten μM of VIP was incubated with varying concentration of SSM or SSMM (lipid:peptide molar ratio ranged from 0 to 40);

FIG. 11 shows the representative volume-weight size distribution of VIP (20 μM)-associated (A) SSM (5 mM) or (B) SSMM (5 mM) using Nicomp; and

FIG. 12 shows the circular dichroism spectra of VIP (20 μM) in [symbol] (a) saline, [symbol] (b) SSM (5 mM) and [symbol] (c) SSMM (5 mM).

### DETAILED DESCRIPTION OF THE INVENTION

While this invention is susceptible of embodiment in many different forms, there is shown in the drawings, and will be described herein in detail, specific embodiments thereof with the understanding that the present disclosure is to be considered as an exemplification of the principles of the invention and is not intended to limit the invention to the specific embodiments illustrated.

The present invention is related generally to compositions of sterically stabilized simple micelles (SSM) of a hydrophilic polymer-conjugated lipid or sterically stabilized mixed micelles (SSMM) of a hydrophilic polymer-conjugated lipid and a water-insoluble lipid. and their use for correcting misfolding of peptides and proteins, which can be used to treat a peptide and protein folding disorder such as such as, but not limited to, alpha-1 antitrypsin deficiency, cystic fibrosis, diabetes type II, hemolytic anemia, Alzheimer's disease for claims because we have data in examples, transmissible spongiform encephalopathies, serpin-deficiency disorders, Huntington disease, Amyotrophic Lateral Sclerosis, Parkinson disease, spinocerebellar ataxias, dialysis-related amyloidosis, polyglutamine diseases, Down's syndrome, Fabry, other gangliosidosis and cataract. The term “misfolding” herein means that the peptide or protein is folding into a conformation other than its native 3-dimensional conformation. Details of protein misfolding have been described by Dobson (Dobson C M. Protein folding and misfolding. Nature. 2003 Dec. 18: 426(6869):884-90; Dobson, C. M., Principles of protein folding, misfolding and aggregation: Seminars in Cell & Dev. Bio. 2004; 15:3-16).

By “peptide and protein folding disorder” is meant a disease or disorder whose pathology is related to the presence of a misfolded protein. In one embodiment, the disorder is caused when a misfolded protein interferes with the normal biological activity of a cell, tissue, or organ. “Peptide and protein folding disorder” is also known as “protein conformational disease”, which, in the present disclosure, are used interchangeably.

The present invention provides a method for treating a peptide and protein folding disorder in a mammalian subject by administering a composition comprising sterically stabilized simple micelles of a hydrophilic polymer-conjugated lipid to the subject or sterically stabilized mixed micelles of a hydrophilic polymer-conjugated lipid and a water-insoluble lipid to the subject. The subject is preferably a human subject. Identifying a subject in need of such treatment can be in the judgment of a subject or a health care professional. The judgment can be subjective (e.g. opinion) or objective (e.g. as determined by a diagnostic test). As used herein, the terms “treat,” “treating,” “treatment,” and the like refer to reducing or ameliorating a disorder and/or symptoms associated therewith. Although not precluded, treating a disorder or condition does not require that the disorder, condition or symptoms associated therewith be completely eliminated. As used herein, the terms “treat,” treating,” “treatment,” and the like may include “prophylactic treatment” which refers to reducing the probability of developing a disorder or condition in a subject, who does not have, but is at risk of or susceptible to developing a disorder or condition.

Hydrophilic polymer-conjugated lipids, such as polyethylene glycol-conjugated (PEGylated) phospholipids, are water soluble and self-assemble as nanosized micelles when their concentrations exceed the critical micelle concentrations (CMC). CMC of the PEGylated phospholipids range from 0.5 to 1.5 μM, with a higher CMC. for longer PEG chain length. These micelles, which are generally less than 100 nm, avoid mononuclear phagocytic system (MPS) uptake and have been demonstrated to have prolonged circulation times (Sethi V. et al., AAPS PharmSci 2003; 5:M1045). They are, therefore, also referred to as sterically stabilized simple micelles (SSM).

SSM according to the present invention may be produced from combinations of lipid materials well known and routinely utilized in the art to produce micelles and including at least one lipid component covalently bonded to a water-soluble polymer. Lipids may include relatively rigid varieties, such as sphingomycelin, or fluid types, such as phospholipids having unsaturated acyl chains, e.g. phosphatidylethanolamine (PE). Polymers of the present invention may include any compounds known and routinely utilized in the art of sterically stabilized liposome (SSL) technology and technologies which are useful for increasing circulatory half-life for proteins, including for example, polyvinyl alcohol, polylactic acid, polyglycolic acid, polyvinylpyrrolidone, polyacrylamide, polyglycerol, polyaxozlines, or synthetic lipids with polymeric head-groups. The most preferred polymer of the invention is polyethylene glycol (PEG) at a molecular weight between 1000 and 5000. Preferred lipids for producing micelles according to the invention include distearoyl-phosphatidylethanolamine covalently bonded to PEG (PEG-DSPE) alone or in further combination with phosphatidylcholine (PC), and phosphatidylglycerol (PG) in further combination with cholesterol (Chol) and/or calmodulin. Methods of preparing sterically stabilized micelles of the present invention can be carried out using various techniques which have been disclosed in details in U.S. Pat. Nos. 6,217,886 and 6,322,810.

SSM of the present invention are dynamic structures. A given SSM system contains micelles in equilibrium with monomeric hydrophilic polymer-conjugated lipids. Not to be bound by any specific theory or hypothesis, it is likely that SSM stabilizes proteins by two mechanisms. First, amphiphilic peptides self-associate with hydrophilic SSM of polymer-conjugated lipids and change their conformation to an active α helix form that results in increased stability of the peptide (Gandhi, S et al., Interactions of human secretin with sterically stabilized phospholipid micelles amplify peptide-induced vasodilation in vivo. Peptides, 2002. 23(8): p. 1433-9; Tsueshita, T., et al., Phospholipids modulate the biophysical properties and vasoactivity of PACAP-(1-38). J Appl Physiol, 2002. 93(4): p. 1377-83; Sethi Varun (2003) PhD Thesis Development and Delivery Of VIP Phospholipid Carriers For the Treatment of Rheumatoid Arthritis University of Illinois at Chicago). Second, monomers of hydrophilic polymer-conjugated lipids can coat the exposed hydrophobic surfaces of proteins and avoid deleterious protein-protein interaction and precipitation in aqueous media. It has been recently proposed that these hydrophobic “hot-spots” are responsible for driving the pathogenesis of several protein misfolding disorders such as AD, Parkinson's and Huntington's disease (Fernandez-Escamilla A et al., Prediction of sequence-dependent and mutational effects on the aggregation of peptides and proteins. Nat Biotechnol 2004; 22(10):1302-6). Therefore, shielding of these hot-spots by hydrophilic polymer-conjugated lipids such as PEGylated phospholipids would prevent their interaction.

SSM may be administered by a route such as, but not limited to, intranasally, intravenously, intra-ventrcularly, intracisternally, subcutaneously, topically, intra-thecally, rectally, vaginally, trans-cutaneously, inhalation, sub-lingually, intra-ocular, ocular or orally. For treating neurodegenerative diseases due to peptide and protein folding disorders, challenges in drug delivery to the brain arise from the presence of one of the most stringent in vivo barriers: the blood-brain barrier (BBB). For peptide and protein folding disorders in the brain, such as Alzheimer disease and Parkinson's disease, SSM are preferably administered intranasally. Several researchers have reported the feasibility of the intranasal route in delivering drug molecules to the brain bypassing the blood brain barrier (Illum L. Is nose-to-brain transport of drugs in man a reality? J Pharm Pharmacol. 2004 January; 56(1):3-17). Research in this field was pioneered by Dr William Frey about two decades ago by publishing a seminal paper (Chen X-Q et al., Delivery of nerve growth factor to the brain via the olfactory pathway. J. Alzheimer's disease 1998; 1(1): 35-44). Thereafter, several researchers reported on the feasibility of this route. It has been shown that that intranasal administration of large proteins such as NGF to a mouse model of AD reaches the brain by bypassing the blood brain barrier (De Rosa R et al., Intranasal administration of nerve growth factor (NGF) rescues recognition memory deficits in AD11 anti-NGF transgenic mice. Proc Natl Acad Sci USA. 2005 Mar. 8; 102(10):3811-6). As shown in Example 3 below, SSM can be uptaken into the brain when administered intranasally. Formulations and preparations of SSM for intranasal delivery are well known to those skill in the art. An example of preparing SSM of the present invention for intranasal delivery is provided in Example 2.

Optionally, the SSM may include a biologically active compound associated with the micelles. Biologically active compounds that can be delivered by SSM are disclosed in detail in U.S. Pat. Nos. 6,218,866 and 6,322,810. The biologically active compounds are preferably amphipathic compounds. What is meant by “amphipathic” is that the compounds have both hydrophilic and hydrophobic portions. The preferred amphipathic compounds are characterized by having hydrophilic domains segregated to the extent that the hydrophobic domain is capable of associating within the micelle core. Examples of a biologically active compound include, but not limited to, vasoactive intestinal peptide (VIP), growth hormone releasing factor (GRF), peptide histidine isoleucine (PHI), peptide histidine methionine (PHM), pituitary adenylate cyclase activating peptide (PACAP), gastric inhibitory hormone (GIP), hemodermin, the growth hormone releasing hormone (GHRH), sauvagine and urotensin I, secretin, glucagon, galanin, endothelin, calcitonin, α₁-proteinase inhibitor, angiotensin II, corticotropin releasing factor, antibacterial peptides and proteins in general, surfactant peptides and proteins, α-MSH, adrenolmedullin, ANF, IGF-1, α2 amylin, orphanin, and orexin.

In addition to the SSM described above, the present invention may also use sterically stabilized mixed micelles (SSMM). Compositions and methods for preparing SSMM are similar to those of SSM except that the micelles further include a water-insoluble lipid, such as a phospholipid, in addition to the hydrophilic polymer-conjugated lipid. A preferred phospholipid as the water-insoluble lipid is phosphatidylcholine. Detail description, compositions, and methods for preparing SSMM have been disclosed previously (U.S. Pat. Nos. 6,217,886 and 6,322,810; Krishnadas A, Rubinstein I, Onyuksel H. Sterically stabilized phospholipid mixed micelles: in vitro evaluation as a novel carrier for water-insoluble drugs. Pharm Res 2003; 20:297-302; Ashok B, Arleth L, Hjelm R P, Rubinstein I, Onyuksel H. In vitro characterization of PEGylated phospholipid micelles for improved drug solubilization: effects of PEG chain length and PC incorporation. J Pharm Sci 2004; 93:2476-87).

The present invention further provides a method for treating Alzheimer's Disease (AD) in a mammalian subject by administering to the subject a composition comprising sterically stabilized simple micelles (SSM) of a hydrophilic polymer-conjugated lipid or sterically stabilized mixed micelles (SSMM) of a hydrophilic polymer-conjugated lipid and a water-insoluble lipid. The subject is preferably a human subject. The SSM and SSMM are described in detail above. The hydrophilic polymer-conjugated lipid is preferably distearoyl phosphatidylethanolamine polyethylene glycol 2000 (DSPE-PEG₂₀₀₀). The water-insoluble lipid is preferably phosphatidylcholine In a preferred embodiment, the composition may further comprise a biologically active agent in association with the SSM or SSMM suitable for treating AD. In an embodiment, the biologically active compound is a member of glucagon/secretin family of peptides, such as, but not limited to, vasoactive intestinal peptide (VIP) and pituitary adenylate cyclase activating peptide (PACAP) wherein the PACAP is a L-isomer or D-isomer. The composition is preferably delivered intranasally.

The present invention still further provides a method for treating Alzheimer's Disease (AD) in a mammalian subject by administering to the subject an effective amount of a composition comprising of a biologically active compound of a member of glucagon/secretin family of peptides associated with the SSM or SSMM or the present invention. Examples of the glucagon/secretin family of peptides include, but not limited to, vasoactive intestinal peptide (VIP) and pituitary adenylate cyclase activating peptide (PACAP), wherein the PACAP is a L-isomer or D-isome. The subject is preferably a human subject. AD is a very distinctive disorder, in that, all the pathophysiological features such as plaque and neuroinflammation coexist at any given point in time. Therefore, targeting only one aspect will not be sufficient for effective AD therapy. Although efforts are underway, treatment of AD still represents an unmet medical need. The present invention of using a combination of SSM and a member of glucagon/secretin family of peptides to treat AD provides a dual therapeutic approach in inhibiting or preventing plaque formation as well as reducing neuroinflammation. As shown in Example 1 below, SSM are able to inhibit Aβ-42 aggregation. The anti-inflammatory properties of glucagon/secretin family of peptides such as VIP, an endogenous neuropeptide, against AD are well established (Gozes I et al., Neuroprotective strategy for Alzheimer disease: intranasal administration of a fatty neuropeptide. Proc Natl Acad Sci USA. 1996 Jan. 9; 93(1):427-32; Delgado, M et al., Vasoactive intestinal peptide prevents activated microglia-induced neurodegeneration under inflammatory conditions: potential therapeutic role in brain trauma. Faseb J, 2003. 17(13): p. 1922-4). However, the rampant usage of these peptides is vastly limited by its in vivo stability issues rendering it ineffectual for further development. Our laboratory has solved this delivery problem by exploiting the innate biophysical properties of these peptides to avidly associate with phospholipid micelles, forming a biocompatible nanoparticular complex possessing extensive therapeutic anti-inflammatory potential (Onyuksel, H., et al., A novel formulation of VIP in sterically stabilized micelles amplifies vasodilation in vivo. Pharm Res, 1999. 16(1): p. 155-60). Conventionally, SSM and SSMM have been explored as drug delivery systems for small molecules or peptide drugs. The present disclosure demonstrates a novel maverick role for SSM and SSMM where they serve dual purposes of: (1) preventing deleterious Aβ aggregation process thereby retarding plaque formation, and (2) delivering a stable biologically active anti-inflammatory peptide at the target tissue where the peptide will elicit its anti-inflammatory property thereby imparting neuroprotection. As discussed earlier, it is likely that SSM or SSMM such as those prepared from PEGylated lipid spontaneously interact with Aβ-42 by two mechanisms: (a) micelles transform Aβ-42 into non-amyloidogenic helical form and (b) hydrophilic polymer-conjugated lipid monomers coat Aβ-42 oligomers and decrease fibril formation. Amelioration of these processes will eventually lead to diminished plaque formation. Anti-inflammatory peptides of the glucagon/secretin family such VIP, having well established neuroprotective and anti-inflammatory activity, serve to mitigate inflammation and provides neuroprotection. Therefore, SSM- or SSMM-VIP (or other members of the glucagon/secrtin family of peptides) formulations possess unique bifunctional therapeutic capabilities targeted towards the two most characteristic hallmarks of AD. Examples of formulations and methods for preparing VIP (and other suitable peptides) associated SSM or SSMM suitable for use in the present invention are disclosed in U.S. Pat. Nos. 6,218,866 and 6,322,810 and by Onyuksel et al. (Onyuksel, H., et al., A novel formulation of VIP in sterically stabilized micelles amplifies vasodilation in vivo. Pharm Res, 1999. 16(1): p. 155-60). Preferably, the formulation is administered to the subject intranasally.

### EXAMPLES

While the following examples are directed to the use of PEGylated phospholipid micelles for the inhibition and the prevention of folding of beta amyloid proteins, the invention is not limited to PEGylated phospholipids. Other hydrophilic polymer-conjugated lipids can be used as discussed earlier. Similarly, the treatment is also not limited to AD, but to any other peptide and protein folding disorders.

### Example 1

### PEGylated Phospholipids Retard β-Amyloid Fibrillogenesis and Confer Neuroprotection

### Statistical Analysis

Data are represented as mean±standard deviation (S.D.) for at least three independent determinations. Difference between groups and its statistical significance was determined using two tailed Student's t-test and ANOVA. All statistical analysis was performed using SPSS version 10.0 (Chicago, Ill.). P value of <0.05 was considered statistically significant.

### Materials

1,2-Distearoyl-sn-glycero-3-phosphoethanolamine-N-methoxy-poly(ethylene glycol 2000) (DSPE-PEG₂₀₀₀) was purchased from Northern Lipids (Vancouver, Canada). Thioflavine T (ThT), Congo Red (CR), 1,1,1,3,3,3-hexafluoro-2-propanol (HFIP) and sodium azide were obtained from Sigma-Aldrich (St. Louis, Mo.). Synthetic Aβ-42 was obtained from American Peptides (Sunnyvale, Calif.). Uranylacetate and other materials required for electron microscopy were purchased from Electron Microscopy Sciences (Hatfield, Pa.). Buffer and all other reagents used were analytical grade and purchased from Sigma-Aldrich. Water was deionized at 18 MΩ and sterile filtered (0.22μ) before use. All peptide and lipid samples were high performance liquid chromatography purified and the peptide purity was always greater than 98% as ascertained by HPLC.

### Preparation of β-Amyloid (Aβ-42)

Stock solution of the peptide was prepared by dissolving the lyophilized peptide in HFIP to a final concentration of 1 mg/ml using a Hamilton syringe equipped with a Teflon plunger (Zagorski M, Yang J, Shao H, Ma K, Zeng H, Hong A. Methodological and chemical factors affecting amyloid beta peptide amyloidogenicity. Methods Enzymol 1999; 309:189-204). This solution was shaken on a Barnstead Lab Line plate shaker for 2 h at 4° C., aliquoted into sterile glass vials, HFIP was removed under vacuum in the fume hood and the peptide was stored desiccated at −20° C. until use (Yoshiike Y, Tanemura K, Murayama O, Akagi T, Murayama M, Sato X, Sun S, Tanaka N, Takashima A. New insights on how metals disrupt amyloid beta-aggregation and their effects on amyloid-beta cytotoxicity. J Biol Chem 2001; 276(34):32293-32299). Prior to use, each vial was allowed to equilibrate at room temperature for 15 min to avoid drastic temperature alteration leading to condensation.

### Preparation of PEGylated Lipid Containing Aβ-42 Samples

The preparation procedure for SSM has been previously disclosed (Gandhi S, Tsueshita T, Onyuksel H, Chandiwala R, Rubinstein I. Interactions of human secretin with sterically stabilized phospholipid micelles amplify peptide-induced vasodilation in vivo. Peptides 2002; 23(8):1433-1439). In the present disclosure, we employed the same protocol with a slight modification. Appropriate amount of DSPE-PEG₂₀₀₀ was added to Aβ-42 in HFIP. This mixture was vortexed for 5 min (Thermolyne Maxi Mix II) and solvent was evaporated to form Aβ-42-lipid film. Residual solvent was removed under vacuum. Films were reconstituted in 10 mM HEPES buffer, vacuum sonicated (Fisher Scientific bath sonicator B2200R-1) and incubated at 25° C. (VWR SHEL LAB Incubator) for 2 h. Films were freshly prepared before each experiment. For SSM and Aβ-42 controls, the same procedure was followed without Aβ-42 and SSM respectively. Size of SSM was ˜14 nm as determined by quasi-elastic light scattering.

### Turbidimetry Assay

Turbidimetry assay was performed as previously described (Jarrett J, Berger E, Lansbury P, Jr. The C-terminus of the beta protein is critical in amyloidogenesis. Ann N Y Acad Sci 1993; 695:144-148) with slight modifications. Samples were prepared as described above (Ashok B, Arleth L, Hjelm R P, Rubinstein I, Onyuksel H. In vitro characterization of PEGylated phospholipid micelles for improved drug solubilization: effects of PEG chain length and PC incorporation. J Pharm Sci 2004; 93(10):2476-2487; Datki Z, Jhász A, Gálfi M, Soós K, Papp R, Zádori D Penke B. Method for measuring neurotoxicity of aggregating polypeptides with the MTT assay on differentiated neuroblastoma cells Brain Research Bulletin 2003; 30; 223-229). For control sample containing Aβ-42 in buffer, same procedure was repeated without lipid. A final Aβ-42 concentration of 25 μM was obtained corresponding to Aβ-42:lipid ratio of 1:0-1:100. Sodium azide (0.01%) was added to the buffer to prevent bacterial contamination. The solution was stirred continuously at room temperature in dark using a magnetic stirrer at ˜60 rpm. Aliquots were withdrawn at pre-defined time intervals in a 96 well plate and shaken for 60 s to evenly resuspend the aggregates. Turbidity was measured at 405 nm using a Labsystems Multiskan Plus UV-Vis Microplate Reader.

### Congo Red (CR) Binding Assay

β-sheet formation of Aβ-42 in presence and absence of lipid was determined by Congo red binding. Aβ-42 (10μM) samples were prepared with or without lipid (0.5 mM) as described above. At the end of 2 h, CR (100 μM stock prepared in NaCl, pH 7.4) was added to the Aβ-42 solution to give a final concentration of 10 μM CR. Solutions were vortexed and incubated at 25° C. for 15 min. Absorbance values at 403 and 541 nm were recorded for samples and CR alone preparations using a Perkin Elmer Lambda 35 UV spectrophotometer in a 1-cm path length cuvette. Background absorbance values of buffer and SSM were subtracted from the respective test solutions. Aggregated Aβ-42 was quantitated as described previously (Klunk W, Jacob R, Mason R. Quantifying amyloid beta-peptide (Abeta) aggregation using the Congo red-Abeta (CR-abeta) spectrophotometric assay. Anal Biochem 1999; 266(1):66-76) using the equation:

Aggregated Aβ(μg/ml)=(^{540nm}A/4780)−(^{403nm}A/6830)−(^{403nm}A_{CR}/8620)

^{540nm} A and ^{403nm} A are absorbance of peptide sample while A_{CR} is the absorbance of CR dye alone. The concentration of aggregated Aβ-42 monomer was then calculated assuming a molecular mass for Aβ-42 of 4514 (obtained from vendor).

### Thioflavine-T (ThT) Binding Assay

The degree of Aβ-42 fibrillization was determined using the fluorescent dye, ThT, which specifically binds to fibrillar conformations (LeVine H, 3rd. Thioflavine T interaction with synthetic Alzheimer's disease beta-amyloid peptides: detection of amyloid aggregation in solution. Protein Sci 1993; 2(3):404-410). Samples were prepared as described above with final Aβ-42 concentration of 25 μM. At the end of 2 h, 200 μL of sample solution was transferred to 96 well Black Cliniplates (Labsystems). ThT was added to each test sample to a final concentration of 10 μM. Samples were shaken for 30 s prior to each measurement. Relative fluorescence intensity was measured using a SpectraMax Gemini XS Plate Reader (Molecular Devices). Measurements were performed at an excitation wavelength of 445 nm and an emission of 481 nm (pre-determined experimentally). To account for background fluorescence, fluorescence intensity from control solution without Aβ-42 was subtracted from solution containing Aβ-42.

### Circular Dichroism Spectroscopy (CD)

Secondary structure of Aβ-42 in presence and absence of lipids were determined by CD spectroscopy. Samples were prepared as described above (10 μM Aβ-42 and peptide:lipid ratio of 1:50) and scanned at room temperature in a 1 mm path length fused quartz cuvette using a Jasco J-710 Spectropolarimeter (Jasco, Easton, Md.) calibrated with d10 camphor sulfonic acid. This service was provided by the Protein Research Lab of Research Resources Center (RRC) of University of Illinois at Chicago. Spectra were obtained from 190-260 nm at 1-nm bandwidth, 5 nm step and 1s response time averaged over 5 runs. Spectra were corrected for buffer or SSM scans and smoothed using manufacturer's Savitzky Golay algorithm. Spectra were deconvoluted and percentage secondary structure was calculated by fitting the data into simulations by SELCON® (Sreerama N, Woody R. Poly (pro)II helices in globular proteins: identification and circular dichroic analysis. Biochemistry 1994; 33(33):10022-10025).

### Particle Size Measurement by Quasi-Elastic Light Scattering

Particle size of aggregates formed by Aβ-42 in presence and absence of lipid were analyzed by quasi-elastic light scattering (QELS) using a NICOMP 380 Particle Size Analyzer (Santa Barbara, Calif.) equipped with a 5 mW helium-neon laser at 632.8 nm and a temperature controlled cell holder. Samples were prepared as described previously. Solutions were stirred continuously at ˜60 rpm at room temperature. 500 μL of test solution was aliquoted after 2 h and particle size distribution of Aβ-42 (12.5 μM; peptide:lipid ratio of 1:50) aggregates was determined. The mean hydrodynamic particle diameter, dₕ was obtained from the Stokes-Einstein relation using the measured diffusion of particles in solution as described previously (Ashok B, Arleth L, Hjelm R P, Rubinstein I, Onyuksel H. In vitro characterization of PEGylated phospholipid micelles for improved drug solubilization: effects of PEG chain length and PC incorporation. J Pharm Sci 2004; 93(10):2476-2487). Data was analyzed in terms of volume weighted distribution.

### Transmission Electron Microscopy (TEM)

The ultrastructural characteristics of Aβ-42 (100 μM) aggregates in presence and absence of lipids were examined under a transmission electron microscope (TEM) (JEOL-JEM 1220, JEOL USA Inc., Peabody, Mass.) at 100 kV for morphology. Use of this equipment was provided Electron Microscopy Services at RRC-UIC. Samples were prepared as described above and incubated at 25 C for 72 h. A 5 μL drop of sample was placed on Formvar carbon support film on copper grid (mesh 200) (Electron Microscopy Sciences, Hatfield, Pa.) stained with 2% uranylacetate for 1 min. Excess stain was removed and samples were dried overnight at room temperature. TEM images were recorded by at 30 000× on a multiscan camera (Gatan Inc., Pleasanton, Calif.) using the Gatan Digital Micrograph version 2.5 software.

### Cytotoxicity Activity

Human Neuroblastoma SHSY-5Y cell line was used to study the effect of PEGylated lipid micelles on Aβ-42 induced toxicity. Cells were cultured in Dulbecco's modified Eagle's medium (DMEM) (Mediatech) supplemented with 4.5 g/L L-glucose, 0.1 mmol/L non essential amino acids, 2 mmol/L glutamine and 10% fetal bovine serum at 37° C. in 5% CO₂. Cells were plated (5×10⁴/well) in 96 well plates in 150 μL of media. After overnight incubation, cells were washed with serum free media. Serum free media alone or containing one of the following combinations (0.2-4 μM of Aβ incubated for 2 h at 25° C. with or without 0.01-0.2 mM of PEGylated lipid; Aβ-42: lipid ratios of 1:50) were added to the cells. Cells were then incubated for further 12 h at 37° C. in 5% CO₂. Cell viability was tested using MTS (3-(4,5-dimethylthiazol-2-yl)-5-(3-carboxymethoxyphenyl)-2-(4-sulfophenyl)-2H-tetrazolium) assay (Cell Titer 96® Aqueous One Solution Cell Proliferation Assay kit; Promega, Madison, Wis.) as described in the manufacturer's protocol. In summary, after the end of incubation period cell media was replaced with 100 μl of RPMI-1640 without phenol red. 20 μL of Cell Titer 96 One® solution reagent was added to each well. The plates were incubated at 37° C. for 3 h in humidified, 5% CO₂ atmosphere. Optical density was then read at 492 nm using a UV Spectrophotometric plate reader (Labsystems) and the values obtained for untreated controls were used to define 100% survival.

### PEGylated Phospholipid Micelles Mitigate β-Sheet Formation and Aggregation of Aβ-42 In Vitro

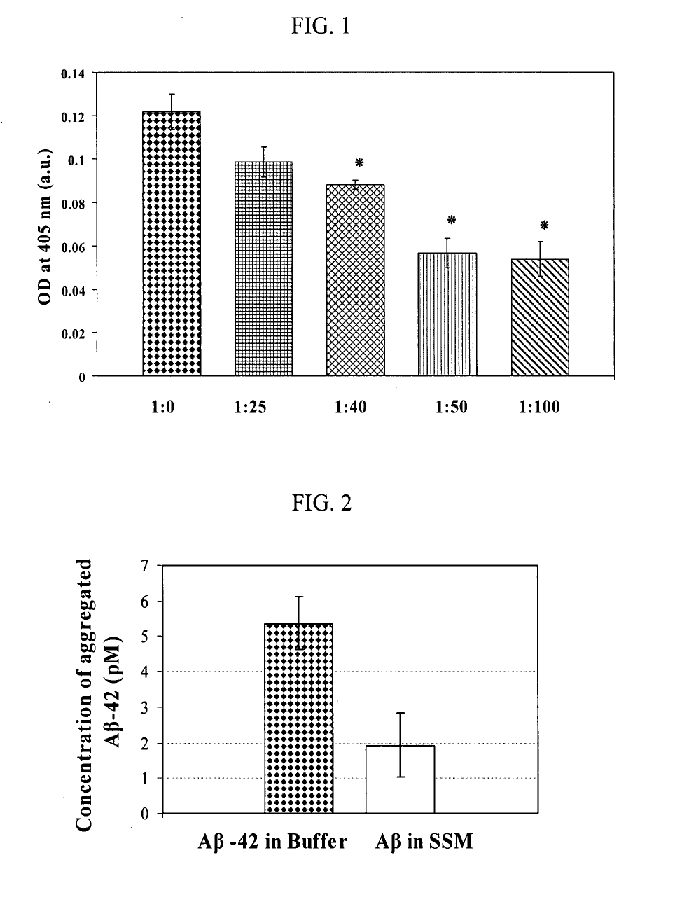

Commercially available synthetic Aβ-42 is usually a heterogeneous mixture of seeds, oligomers and fibrils. To ensure sample homogeneity, HFIP pre-treatment was carried out, thereby facilitating the examination of the effect of PEGylated phospholipid micelles on Aβ-42 aggregation in a more physiologically relevant state. A pilot turbidimetric study was performed to obtain the optimal peptide to lipid (P/L) ratio at which significant inhibition of aggregation was observed. Aβ-42 (25 μM) was incubated with five P/L ratios ranging from 1:25 to 1:100 for 2 h at 25° C. and optical density (OD) measurements were carried out at 405 nm. OD values (FIG. 1) demonstrate a significant retardation in the extent of Aβ-42 aggregation of lipid treated peptide at 1:40, 1:50 and 1:100 P/L ratios. However, saturation was observed at P/L 1:50. Aggregation inhibitory efficacy was not significantly different for P/L ratios of 1:50 and 1:100 and therefore, 1:50 was chosen for further exploratory studies. This value is in good agreement with the value of 1:55 reported previously for Aβ-40 using a lipid bilayer archetype (Terzi E, Holzemann G, Seelig J. Interaction of Alzheimer beta-amyloid peptide (1-40) with lipid membranes. Biochemistry 1997; 36(48):14845-14852). However, turbidity measurement at 405 nm, per se, is a generic aggregation assay that is not conclusive for detection of amyloid fibrillization process. Therefore, we employed more specific deterministic techniques such as Congo red binding and Thioflavine-T interaction assay to obtain fundamental information regarding the nature of effect of PEGylated lipid micelles on Aβ-42 aggregation.

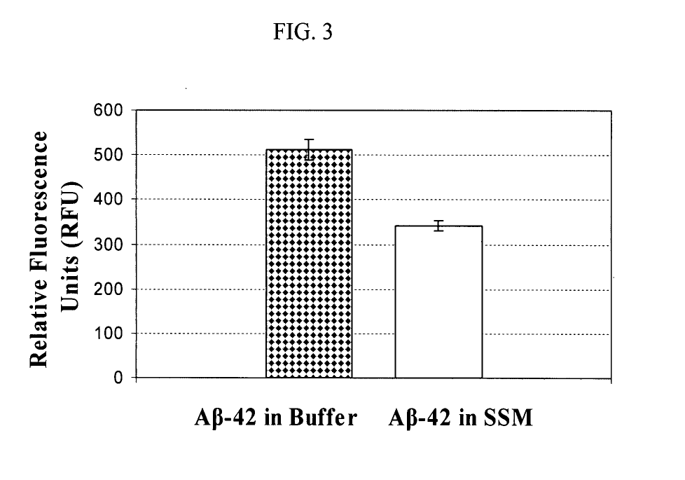

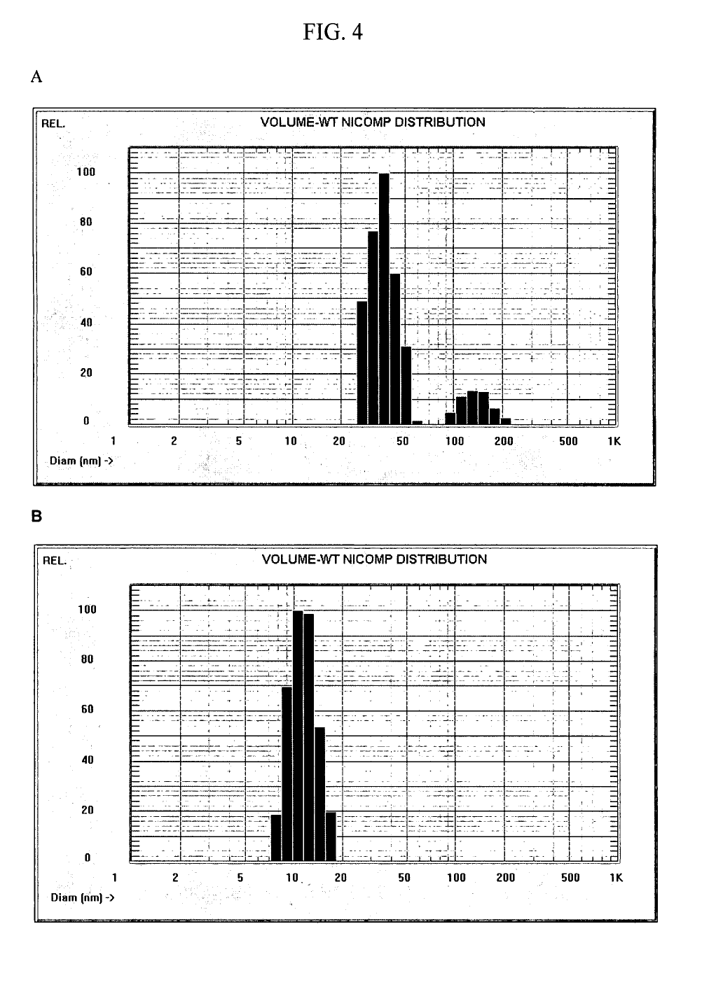

In general, amyloid protein fibrils possess tinctorial dye binding properties owing to their characteristic fibrillar conformations. ThT and CR are two standard dyes used to monitor fibrillogenesis. Binding of ThT to amyloid fibrils causes enhancement of ThT fluorescence, while binding to CR causes a red shift in the absorbance spectrum of the dye and golden birefringence of aggregates under polarized light. We used CR binding assay to quantify the concentration of aggregated β-sheeted amyloid as described previously (Klunk W, Jacob R, Mason R. Quantifying amyloid beta-peptide (Abeta) aggregation using the Congo red-Abeta (CR-abeta) spectrophotometric assay. Anal Biochem 1999; 266(1):66-76). ThT assay was used for semi-quantitative determination of extent of fibril formation. The results of CR binding assay demonstrated that concentration of aggregated β-sheeted Aβ-42 in PEGylated lipid treated sample was reduced almost 3 fold (˜1.9 pM) (p<0.05) compared to untreated control (˜5.8 pM) (FIG. 2). ThT fluorescence spectroscopic assay was then employed to confirm this observation and complementary results were obtained. Relative fluorescence intensity of PEGylated lipid treated sample was significantly lower than that of untreated control, indicating significant mitigation of β-sheeted fibril formation in lipid treated samples (FIG. 3).

We postulated that PEGylated phospholipid micelles retard Aβ-42 aggregation by inducing a constructive conformational change in its secondary structure. CD spectroscopy was performed to obtain a qualitative estimate of Aβ-42 secondary structure in the presence of PEGylated phospholipid micelles. CD scans were deconvoluted using SELCON® software to obtain percentage of each secondary structural element. After incubation of the peptide in buffer for 2 h at 25° C., Aβ-42 exhibited ˜38% β-sheeted conformation while α-helicity was insignificant (˜1.9%) indicating an onset of aggregation. However, upon incubation with PEGylated lipid micelles, a radical alteration in the relative proportions of secondary structural elements was observed. In presence of PEGylated lipid micelles, folding of Aβ-42 was significantly changed resulting in very high proportions of α-helicity (˜34%) and a concurrent favorable decline in β-sheet conformation (˜3.4%) (Table 1). Therefore, it is evinced that in presence of PEGylated lipid micelles, transformation of Aβ-42 to pathogenic β-sheets is significantly inhibited and α-helicity is radically enhanced compared to respective untreated Aβ-42 control. This change in the secondary structure of the peptide in presence of PEGylated lipid micelles is directly responsible for reduction in the Aβ-42 aggregation rate.

Results obtained from CD study concur well with the CR binding and ThT assay which demonstrated that a significant reduction in β-sheeted fibrillar conformation is obtained on treatment with PEGylated phospholipid micelles.

TABLE 1
Influence of PEGylated lipid micelles on Aβ-42
secondary structure by circular dichroism.

| | | Aβ-42 in |
|---|---|---|
| | Aβ-42 in buffer^{§} | SSM* |
| % α-Helix | 1.9 ± 0.25 | 34.25 ± 0.75 |
| % β-Sheet | 38.1 ± 1 | 3.4 ± 0.5 |

Data represents average of 5 accumulations. (*p < 0.05 compared to ^{§})

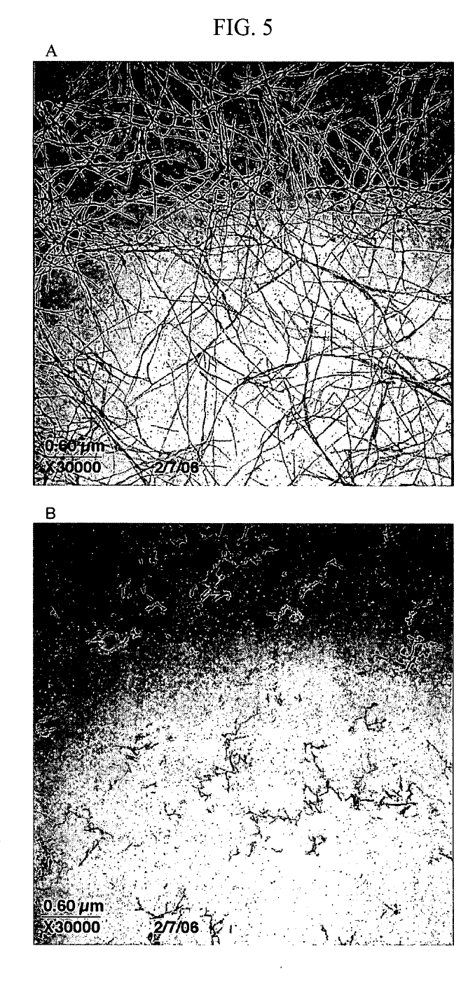

We speculated that the ability of PEGylated lipid micelles to attenuate Aβ-42 aggregation could also manifest in reduction of Aβ-42 aggregate size. To obtain comprehensive information on representative dimensions of Aβ-42 aggregates, quasi-elastic light scattering was employed. After incubation of Aβ-42 (12.5 μM) in buffer for 2 h, a heterogeneous distribution with dual peaks having a maximum average hydrodynamic diameter of 134.4 nm was observed (FIG. 4A). However, in presence of PEGylated phospholipid micelles, the particle size distribution was more homogenous and stable with a single peak at ˜12 nm corresponding to the size of PEGylated phospholipid micelles (FIG. 4B).

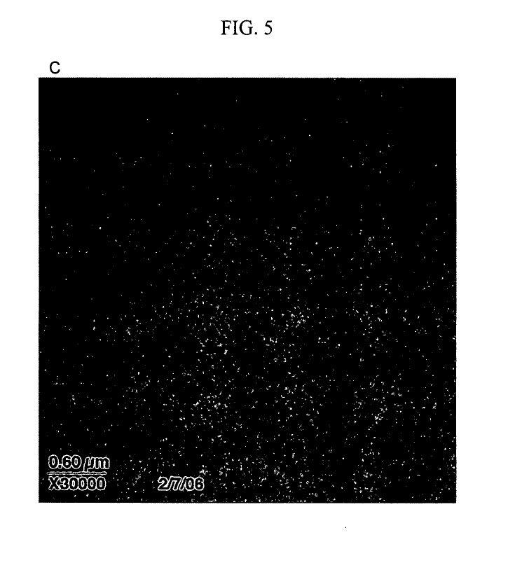

Transmission electron microscopy was employed to determine the effect of PEGylated phospholipid micelles on ultrastructure of Aβ-42 aggregates. Solutions of Aβ-42 (100 μM) were incubated at 25° C. with or without PEGylated lipid micelles. After 72 h, samples were placed on copper grids, negatively stained with 2% uranylacetate and visualized under TEM. In absence of PEGylated phospholipid micelles Aβ-42 formed a dense meshwork of elongated fibrils that covered the entire grid area (FIG. 5A). Presence of micelles ameliorated fibril growth significantly and much shorter fragments were formed (FIG. 5B). The density of these short fragments on each copper grid was much sparse compared to lipid untreated controls.

### PEGylated Lipid Micelles Attenuate Neurotoxicity of Aβ-42 In Vitro

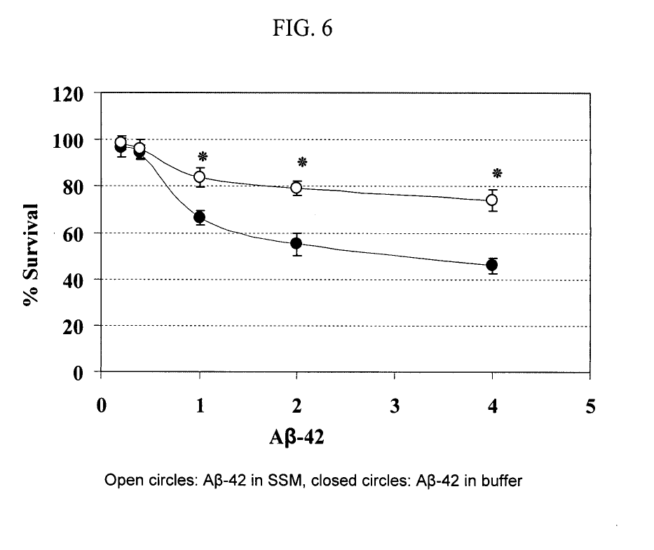

Aβ-42 is shown to be toxic to neurons and cause cell death via apoptotic mechanisms (Allen J, Eldadah B, Huang X, Knoblach S, Faden A. Multiple caspases are involved in beta-amyloid-induced neuronal apoptosis. J Neurosci Res 2001; 65(1):45-53). MTS assay provides a good estimate of cell survival based on bioreduction of MTS to aqueous soluble colored formazan crystals accomplished by dehydrogenase enzymes found in metabolically active cells. Cytotoxicity study was carried out using human neuroblastoma SHSY-5Y cell paradigm that possess highly developed neurites and exhibit high sensitivity against Aβ-42 (Datki Z, Jhász A, Gálfi Soós K, Papp R, Zádori D Penke B. Method for measuring neurotoxicity of aggregating polypeptides with the MTT assay on differentiated neuroblastoma cells Brain Research Bulletin 2003 30; 223-229). A series of physiologically relevant Aβ-42 concentrations (0.2 μM-4 μM) were tested. Lipid untreated Aβ-42 demonstrated elevated neurotoxicity above 1 μM concentration. However, when incubated with PEGylated phospholipid micelles, Aβ-42 neurotoxicity was significantly mitigated and percentage survival was increased by almost 30% compared to lipid untreated control (FIG. 6).

### Discussion

At least 16 different proteins have been identified hitherto that have a high propensity to misfold and form β-sheeted amyloid fibrils leading to toxic gain of function and associated pathologies. Structural context plays a critical role in protein conformational change, their subsequent misfolding and dysregulation. It has been reported that amyloidogenic peptides and proteins contain short stretches of amino acid sequences referred to as “hot spots” that facilitate and drive this aggregation process (Fernandez-Escamilla A, Rousseau F, Schymkowitz J, Serrano L. Prediction of sequence-dependent and mutational effects on the aggregation of peptides and proteins. Nat Biotechnol 2004; 22(10):1302-1306). In its native state, Aβ-42 “hot spots” are stabilized in α-helical conformation by the cell membrane bilayer (Schroeder F, Jefferson J, Kier A, Knittel J, Scallen T, Wood W, Hapala I. Membrane cholesterol dynamics: cholesterol domains and kinetic pools. Proc Soc Exp Biol Med 1991; 196(3):235-252). Therefore, a promising therapeutic strategy to prevent aggregation would be to stabilize this native state of the peptide and coat the “hot spots” by providing a steric barrier to prevent their interaction (Dobson C M. Protein folding and misfolding. Nature. 2003 Dec. 18: 426(6869):884-90). The objective of this study was to test the hypothesis that PEGylated lipid micelles mitigate Aβ-42 aggregation by providing a cell membrane simulating milieu that constrains the peptide in a favorable α-helical conformation preventing its conversion to pathogenic β-sheet form. The lipid monomers (which are in dynamic equilibrium with the micelles) coat the exposed “hot spots” reducing any further deleterious peptide-peptide interaction. The rationale behind this hypothesis was based on our previous experience with several amphiphilic peptides and proteinsm (Gandhi S, Tsueshita T, Onyuksel H, Chandiwala R, Rubinstein I. Interactions of human secretin with sterically stabilized phospholipid micelles amplify peptide-induced vasodilation in vivo. Peptides 2002; 23(8):1433-1439; Kirchoff C, Rubinstein I, Ludwig J, Onyuksel H., DSPE-PEG5000 Increases Physical Stability Of Human IL-2 In vitro (2001) Proceedings Controlled Release Of Bioactive Materials 28:524-525; Onyuksel H, Ikezaki H, Patel M, Gao X P, Rubinstein I. A novel formulation of VIP in sterically stabilized micelles amplifies vasodilation in vivo. Pharm Res 1999; 16(1):155-160; Tsueshita T, Gandhi S, Onyuksel H, Rubinstein I. Phospholipids modulate the biophysical properties and vasoactivity of PACAP-(1-38). J Appl Physiol 2002; 93(4):1377-1383). and on the observation that Aβ structure examined in membrane mimicking surfactants and organic solvents resembled the native non-pathogenic α-helical structure of transmembrane Aβ in vivo (Shao H, Jao S, Ma K, Zagorski M. Solution structures of micelle-bound amyloid beta-(1-40) and beta-(1-42) peptides of Alzheimer's disease. J Mol Biol 1999; 285(2):755-773; Zagorski M, Barrow C. NMR studies of amyloid beta-peptides: proton assignments, secondary structure, and mechanism of an alpha-helix----beta-sheet conversion for a homologous, 28-residue, N-terminal fragments. Biochemistry 1992; 31(24):5621-5631).

### Example 2

### Preparation of SSM for Intranasal Delivery

SSM can be prepared by weighing dried lipid DSPE-PEG₂₀₀₀ in a clean sterile vial. Dry lipid powder (2.2, 5.5 and 11 mM) is weighed and added to a sterile vial following which it is hydrated with 1.0 ml of 10 mM isotonic PBS (pH 7.4). The dispersion is vortexed vigorously for 5 min to homogenize, suspend and dissolve the lipid in the vial. Following this, the dispersion is bath sonicated for 10 min. SSM is formed spontaneously. Intranasal administration can be performed using, for example, a nasal instillation method as described earlier (De Rosa R et al., Intranasal administration of nerve growth factor (NGF) rescues recognition memory deficits in AD11 anti-NGF transgenic mice. Proc Natl Acad Sci USA. 2005 Mar. 8; 102(10):3811-6).

### Example 3

### Brain Uptake of Intranasally Delivered SSM-Quantum Dot (QD)

We performed a study to determine if intranasally administered SSM-QD reached the brain. SSM-QD was prepared as described earlier (Rubinstein I et al., Proc. FASEB 179.8 (2005)) (Rubinstein, 2005) with 5 mM total lipids and 254 of Cd/Se Zn QD (2 mg/ml) (Evident Tech.). Normal Balb/C6 mice were anaesthetized with ketamine/xylazine (90 mg/3 mg/kg of body weight) and 120 uL of SSM-QD was administered intranasally as described earlier (De Rosa R et al., Intranasal administration of nerve growth factor (NGF) rescues recognition memory deficits in AD11 anti-NGF transgenic mice. Proc Natl Acad Sci USA. 2005 Mar. 8; 102(10):3811-6).

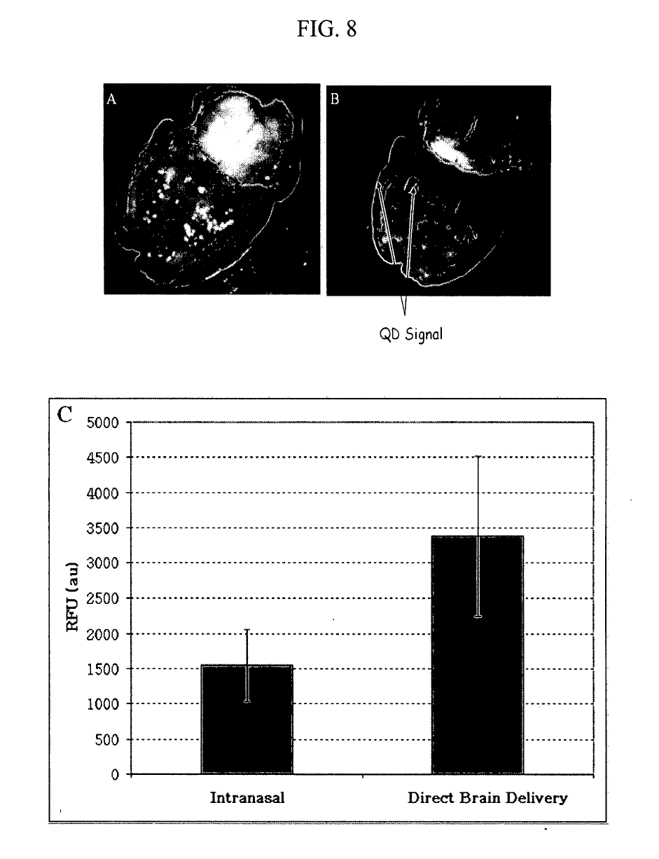

Mice were sacrificed and brain was isolated out and photographed under a hand held UV lamp. For control samples, mice were sacrificed and brain was dissected out. 120 uL of SSM-QD was directly injected. Brain sections were then homogenized in a tissue homogenizer with 1 ml of 0.1M NaOH to extract out the quantum dots. Samples were incubated for 2 h at 4° C. and centrifuged at 13000×G for 10 min. Relative fluorescent intensity of supernatant was analyzed in a spectrofluorometer at excitation of 599 nm and emission of 621 nm (as per QD manufacturers specification). When held under a UV lamp, QD fluorescence was observed. On quantification of fluorescence, it was observed that ˜45% of the dose reached the brain via intranasal route (FIG. 8). These data, although preliminary, provide promising evidence for the nose to brain delivery of SSM.

### Example 4

### Effect of SSM and SSMM on Stability of VIP

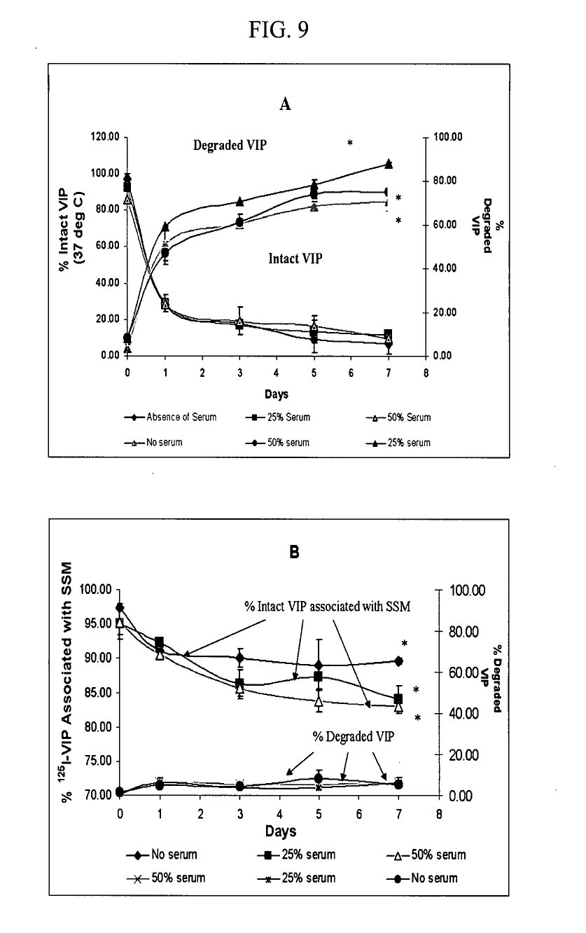

In the specific aim 1.2 of this application, we propose to test the neuroprotective activity of SSM-VIP against neuroinflammation. As mentioned earlier, anti-inflammatory effect of VIP against AD has been well established. However, clinical use of VIP is limited by its susceptibility to degradation in vivo resulting in a half life of few minutes. Our laboratory has solved this complex problem by using SSM as delivery system for VIP. We have previously shown that incubation of VIP with SSM led to a significant enhancement in the stability of VIP in vitro as well as in an animal model (Sejourne, F., et al., Development of a novel bioactive formulation of vasoactive intestinal peptide in sterically stabilized liposomes. Pharm Res, 1997. 14(3): p. 362-5; Sethi Varun (2003) PhD Thesis Development and Delivery Of VIP Phospholipid Carriers For the Treatment of Rheumatoid Arthritis University of Illinois at Chicago) and also demonstrated that the self-association of VIP with SSM also imparted improved bioactivity of VIP (Onyuksel, H., et al., A novel formulation of VIP in sterically stabilized micelles amplifies vasodilation in vivo. Pharm Res, 1999. 16(1): p. 155-60). These results are explained by the conformational change from unstable random coil in aqueous solution to a more stable and active α-helical form that occur in the VIP in the presence of the preferable lipid environment provided by the micelles (Rubinstein I et al., Proc FASEB 179.8 (2005)). Table 2 demonstrates the increase in the α-helicity and circulation half life of VIP in presence of SSM. Since VIP is highly susceptible to enzymatic hydrolysis in serum, we have also tested serum stability of SSM-VIP formulation in a surrogate in vitro system. Briefly, DSPE-PEG₂₀₀₀ (5 mM) was weighed and hydrated (10 mM HEPES buffer, pH 7.4). VIP (5 nmol) was added to preformed SSM and incubated for 2 h at 25° C. to form VIP-SSM. Formulation was then incubated in human serum (25, 50% v/v). Sample aliquots were removed and analyzed on 0, 1, 3, 5 and 7 days following storage at 37° C. These samples were analyzed for the % of intact VIP associated with SSM following separation of unbound VIP from SSM. Results indicated that ˜65% of native VIP in buffer was degraded within 24 h (FIG. 9A). However, on the other hand similar experiments conducted using α-helix VIP (5 mM DSPE-PEG-₂₀₀₀+5 nmol VIP) samples stored in the presence of human serum demonstrated that the passive association of VIP with SSM (α-helix VIP) significantly stabilized the formulation, reducing the % VIP that was degraded in the presence of human serum (up to 50%) (FIG. 9B). This result was most likely due to the association of VIP with the palisade region of the micelles, thereby allowing the PEG to function as a brush border and hindering both the opsonization and interaction of the proteases and serum components from binding with the micelles. Therefore reduced access of the endopeptidases and proteases to the micelles allowed for greater % of the peptide associated with the micelles to remain in the intact form (˜85% of VIP after 7 days at 37° C.).

TABLE 2
Characteristics of VIP (α-helicity and in vivo
half life) in saline and SSM

VIP

| | Saline | SSM |
|---|---|---|
| % α-Helix | 5 ± 1 | 27 ± 2 |
| Circulating t½ in vivo (hours) | 0.3 | 9.6 |

SSMM-VIP formulation can be similarly prepared by including phosphatidylcholine according to Ashok et al. (Ashok B et al., J. Pharm Sci 2004; 93:2476-2487). Table 3 is a summary of the comparison of physical properties of VIP in association with SSM or SSMM.

TABLE 3
Comparison of physical properties of VIP in
association with SSM or SSMM.

| | | Lipid:peptide | # of | Particle | |
|---|---|---|---|---|---|
| | Micellar | saturation | peptide/ | size | |
| Peptide | system | ratio | micelle | (nm) | Anisotropy |
| VIP | Saline | — | — | — | 0.052 ± 0.004 |
| | SSM | 39.9 ± 7.3 | 2.3 ± 0.4 | 14.3 ± 2.4 | 0.152 ± 0.002 |
| | SSMM | 43.3 ± 3.6 | 2.1 ± 0.2 | 13.9 ± 2.3 | 0.148 ± 0.002 |

Lipid:peptide saturation ratio and number of peptide/micelle were determined via fluorescent spectroscopy where 10 μM of VIP was incubuated with varying concentration of SSM or SSMM to achieve lipid:peptide molar ratio ranging from 0 to 40. The data was then fitted into a simple, rectangular hyperbola curve using SigmaPlot® to determine lipid:peptide saturation ratio. The maximum number of peptide molecules that could interact with each micelle was calculated from the aggregation number of lipid monomers per micelle (˜90) for both SSM and SSMM (Ashok B, et al. J Pharm Sci 2004; 93: 2476-2487).
α-helicity was determined by CD using 20 μM of VIP in 5 mM of SSM or SSMM (lipid:VIP ratio of 250:1). The same samples were used for particle size measurement.
Fluorescent anisotropy was conducted using 100 μM of VIP in 4.5 mM of SSM or SSMM (lipid:VIP ratio of 45:1) which is close to the lipid:peptide saturation ratio. The measurements were done using Perkin Elmer luminescence spectrometer LS50B.

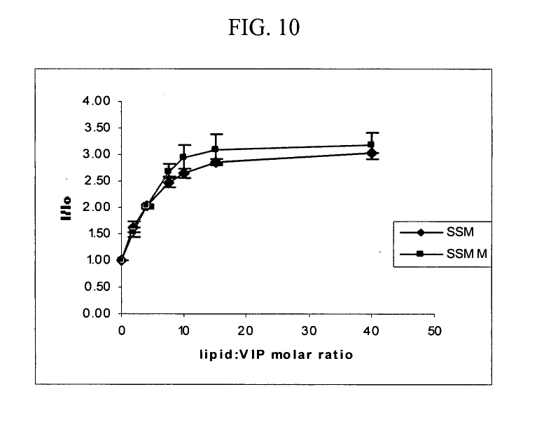

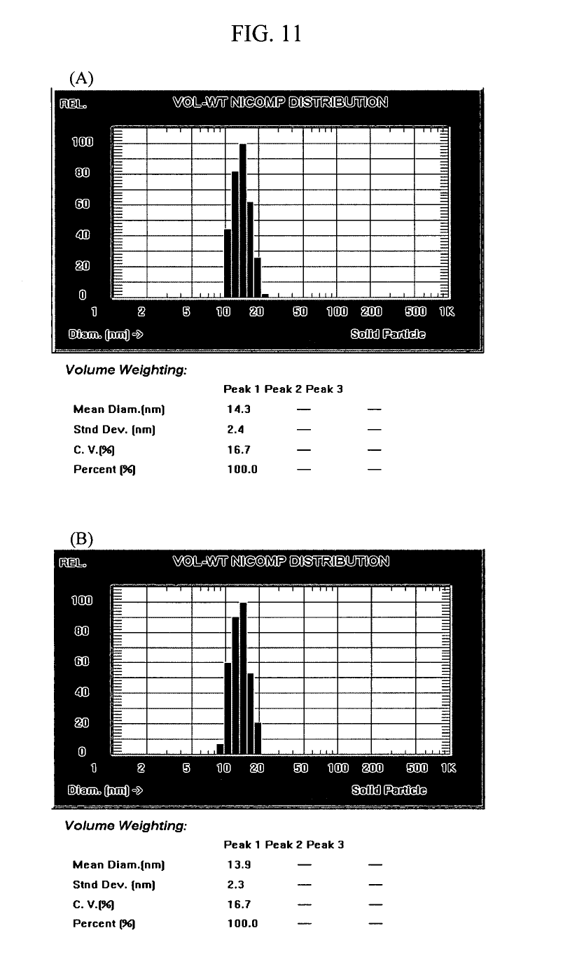

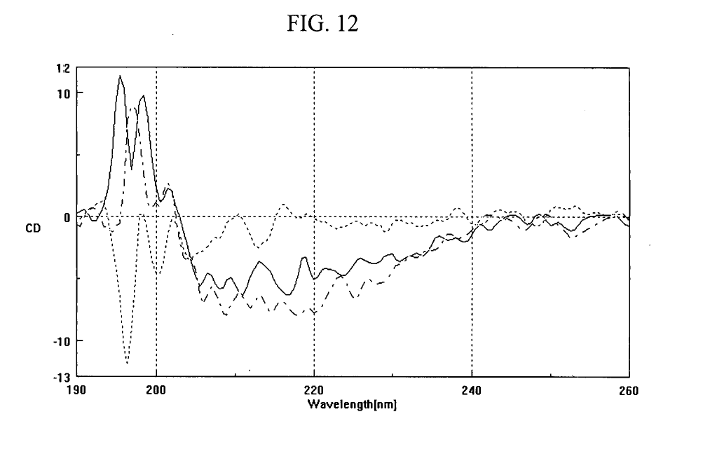

FIG. 10 are lipid:VIP saturation curves in SSM and SSMM determined using fluorescent spectroscopy. Ten μM of VIP was incubuated with varying concentration of SSM or SSMM (lipid:peptide molar ratio ranged from 0 to 40). FIG. 11 is a representative volume-weight size distribution of VIP (20 μM)-associated (A) SSM (5 mM) or (B) SSMM (5 mM) using Nicomp. FIG. 12 are circular dichroism spectra of VIP (20 μM) in (a) saline, (b) SSM (5 mM) and (c) SSMM (5 mM).

### Example 5

### Preparation of SSM-VIP Formulation for Intranasal Delivery

SSM-VIP formulation for intranasal delivery can be prepared by weighing dried lipid DSPE-PEG₂₀₀₀ in a sterile vial. The weight of DSPEPEG₂₀₀₀ is equal to that required for stabilizing VIP (1:40 peptide:lipid saturation ratio). Lipid is hydrated with 1.0 ml of 10 mM isotonic PBS (pH 7.4). The dispersion is vortexed vigorously for 5 min to homogenize, suspend and dissolve the lipid in the vial. Following this, the dispersion is bath sonicated for 10 min. SSM is formed spontaneously. Since VIP is amphiphilic, it is passively associated with the amphiphilic phospholipid, allowing for spontaneous loading into preformed micelles. VIP (VIP dose in lyophilized form is weighed, mixed with preformed micelles and the mixture is allowed to incubate at 25° C. to bring about equilibrium. To this SSM-VIP, appropriately weighed additional SSM is added and allowed to incubate for 1 h. The final formulation contains SSM-VIP plus SSM to exert anti-inflammatory and anti-aggregation effect respectively.

The practice of the present invention will employ and incorporate, unless otherwise indicated, conventional techniques of cell biology, cell culture, molecular biology, microbiology, genetic engineering, and immunology, which are within the skill of the art. While the present invention is described in connection with what is presently considered to be the most practical and preferred embodiments, it should be appreciated that the invention is not limited to the disclosed embodiments, and is intended to cover various modifications and equivalent arrangements included within the spirit and scope of the claims. Modifications and variations in the present invention may be made without departing from the novel aspects of the invention as defined in the claims. The appended claims should, be construed broadly and in a manner consistent with the spirit and the scope of the invention herein.

### REFERENCES

Allen J, Eldadah B, Huang X, Knoblach S, Faden A. Multiple caspases are involved in beta-amyloid-induced neuronal apoptosis. J Neurosci Res 2001; 65(1):45-53.
Ashok B, Arleth L, Hjelm R P, Rubinstein I, Onyuksel H. In vitro characterization of PEGylated phospholipid micelles for improved drug solubilization: effects of PEG chain length and PC incorporation. J Pharm Sci 2004; 93(10):2476-2487.
Datki Z, Jhász A, Gálfi M, Soós K, Papp R, Zádori D Penke B. Method for measuring neurotoxicity of aggregating polypeptides with the MTT assay on differentiated neuroblastoma cells Brain Research Bulletin 2003 30; 223-229.
Delgado, M et al., Vasoactive intestinal peptide prevents activated microglia-induced neurodegeneration under inflammatory conditions: potential therapeutic role in brain trauma. Faseb J, 2003. 17(13): p. 1922-4.
De Rosa R et al., Intranasal administration of nerve growth factor (NGF) rescues recognition memory deficits in AD11 anti-NGF transgenic mice. Proc Natl Acad Sci USA. 2005 Mar. 8; 102(10):3811-6.
Dobson C M. Protein folding and misfolding. Nature. 2003 Dec. 18; 426(6968):884-90.
Dobson, C. M., Principles of protein folding, misfolding and aggregation: Seminars in Cell & Dev. Bio. 2004; 15:3-16.
Fernandez-Escamilla A, Rousseau F, Schymkowitz J, Serrano L. Prediction of sequence-dependent and mutational effects on the aggregation of peptides and proteins. Nat Biotechnol 2004; 22(10):1302-1306.
Gandhi S, Tsueshita T, Onyuksel H, Chandiwala R, Rubinstein 1. Interactions of human secretin with sterically stabilized phospholipid micelles amplify peptide-induced vasodilation in vivo. Peptides 2002; 23(8):1433-1439.
Geerts H. NC-531 (Neurochem). Curr Opin Investig Drugs. 2004 January; 5(1):95-100.
Gandhi, S et al., Interactions of human secretin with sterically stabilized phospholipid micelles amplify peptide-induced vasodilation in vivo. Peptides, 2002. 23(8): p. 1433-9.
Gozes I et al., Neuroprotective strategy for Alzheimer disease: intranasal administration of a fatty neuropeptide. Proc Natl Acad Sci USA. 1996 Jan. 9; 93(1):427-32.
Haass C, Selkoe D. Alzheimer's disease. A technical KO of amyloid-beta peptide. Nature 1998; 391(6665):339-340
Hardy J, Selkoe D. The amyloid hypothesis of Alzheimer's disease: progress and problems on the road to therapeutics. Science 2002; 297(5580):353-356.
Ilium L. Is nose-to-brain transport of drugs in man a reality? J Pharm Pharmacol. 2004 January; 56(1):3-17
Jarrett J, Berger E, Lansbury P, Jr. The C-terminus of the beta protein is critical in amyloidogenesis. Ann N Y Acad Sci 1993; 695:144-148.
Kirchoff C, Rubinstein I, Ludwig J, Onyuksel H., DSPE-PEG5000 Increases Physical Stability Of Human IL-2 In vitro (2001) Proceedings Controlled Release Of Bioactive Materials 28:524-525.
Klunk W, Jacob R, Mason R. Quantifying amyloid beta-peptide (Abeta) aggregation using the Congo red-Abeta (CR-abeta) spectrophotometric assay. Anal Biochem 1999; 266(1):66-76.
Kohno T, Kobayashi K, Maeda T, Sato K, Takashima A. Three-dimensional structures of the amyloid beta peptide (25-35) in membrane-mimicking environment. Biochemistry 1996; 35(50):16094-16104.
Krishnadas A, Rubinstein 1, Onyuksel H. Sterically stabilized phospholipid mixed micelles: in vitro evaluation as a novel carrier for water-insoluble drugs. Pharm Res 2003; 20:297-302.
LeVine H, 3rd. Thiofiavine T interaction with synthetic Alzheimer's disease beta-amyloid peptides: detection of amyloid aggregation in solution. Protein Sci 1993; 2(3):404-410.
Lorenzo A, Yankner B. Beta-amyloid neurotoxicity requires fibril formation and is inhibited by congo red. Proc Natl Acad Sci USA 1994; 91(25):12243-12247.
Masters C, Simms G, Weinman N, Multhaup G, McDonald B, Beyreuther K. Amyloid plaque core protein in Alzheimer disease and Down syndrome. Proc Natl Acad Sci USA. 1985; 82(12):4245-4249.
Onyuksel H, Ikezaki H, Patel M, Gao X P, Rubinstein I. A novel formulation of VIP in sterically stabilized micelles amplifies vasodilation in vivo. Pharm Res 1999; 16(1):155-160.
Pervushin K, Orekhov V, Popov A, Musina L, Arseniev A. Three-dimensional structure of (1-71) bacterioopsin solubilized in methanol/chloroform and SDS micelles determined by 15N-1H heteronuclear NMR spectroscopy. Eur J Biochem 1994; 219(1-2):571-583.
Rizo J, Blanco F, Kobe B, Bruch M, Gierasch L. Conformational behavior of Escherichia coli OmpA signal peptides in membrane mimetic environments. Biochemistry 1993; 32(18):4881-4894.
Rubinstein I et al., Proc. FASEB 179.8 (2005).
Roher A, Lowenson J, Clarke S, Woods A, Cotter R, Gowing E, Ball M. beta-Amyloid-(1-42) is a major component of cerebrovascular amyloid deposits: implications for the pathology of Alzheimer disease. Proc Natl Acad Sci USA 1993; 90(22):10836-10840.
Sabate R, Estelrich J. Stimulatory and inhibitory effects of alkyl bromide surfactants on beta-amyloid fibrillogenesis. Langmuir. 2005 Jul. 19; 21(15):6944-9.
Schroeder F, Jefferson J, Kier A, Knittel J, Scallen T, Wood W, Hapala I. Membrane cholesterol dynamics: cholesterol domains and kinetic pools. Proc Soc Exp Biol Med 1991; 196(3):235-252.
Selkoe D. Alzheimer's disease: genes, proteins, and therapy. Physiol Rev 2001; 81(2):741-766.

Sejourne, F., et al., Development of a novel bioactive formulation of vasoactive intestinal peptide in sterically stabilized liposomes. Pharm Res, 1997. 14(3): p. 362-5Serpell L. Alzheimer's amyloid fibrils: structure and assembly. Biochim Biophys Acta 2000; 1502(1):16-30.
Sethi V., Onyuksel H., Rubenstein I. Enhanced circulation half-life and reduced clearance of vasoactive intestine peptide (VIP) loaded in sterically stabilized micelles (ZSSM) in mice with collagen-induced arthritis (CIA), AAPS PharmSci 2003; 5:M1045, Lukyanov A N et al. Pharm Res 2002; 19:1424-9.
Sethi Varun (2003) PhD Thesis Development and Delivery Of VIP Phospholipid Carriers For the Treatment of Rheumatoid Arthritis University of Illinois at Chicago.
Shao H, Jao S, Ma K, Zagorski M. Solution structures of micelle-bound amyloid beta-(1-40) and beta-(1-42) peptides of Alzheimer's disease. J Mol Biol 1999; 285(2):755-773.
Smith M, Drew K, Nunomura A, Takeda A, Hirai K, Zhu X, Atwood C, Raina A, Rottkamp C, Sayre L, Friedland R, Perry G. Amyloid-beta, tau alterations and mitochondrial dysfunction in Alzheimer disease: the chickens or the eggs? Neurochem Int 2002; 40(6):527-531.
Sreerama N, Woody R. Poly (pro)II helices in globular proteins: identification and circular dichroic analysis. Biochemistry 1994; 33(33):10022-10025.
Terzi E, Holzemann G, Seelig J. Interaction of Alzheimer beta-amyloid peptide (1-40) with lipid membranes. Biochemistry 1997; 36(48):14845-14852.
Tsueshita T, Gandhi S, Onyuksel H, Rubinstein I. Phospholipids modulate the biophysical properties and vasoactivity of PACAP-(1-38). J Appl Physiol 2002; 93(4):1377-1383
Walsh D, Klyubin G, Shankar G, Townsend M, Fadeeva J, Betts V, Podlisny M, Cleary J, Ashe K, Rowan M, Selkoe D. The role of cell derived oligomers of Aβ in Alzheimer's disease and avenues for therapeutic intervention. Proteins in Disease, Biochemical Society Transactions 2005; 33(5):1087-090.
Waterhous D, Johnson W, Jr. Importance of environment in determining secondary structure in proteins. Biochemistry 1994; 33(8):2121-2\128.
Yoshiike Y, Tanemura K, Murayama O, Akagi T, Murayama M, Sato X, Sun S, Tanaka N, Takashima A. New insights on how metals disrupt amyloid beta-aggregation and their effects on amyloid-beta cytotoxicity. J Biol Chem 2001; 276(34):32293-32299.
Zagorski M, Barrow C. NMR studies of amyloid beta-peptides: proton assignments, secondary structure, and mechanism of an alpha-helix----beta-sheet conversion for a homologous, 28-residue, N-terminal fragments. Biochemistry 1992; 31(24):5621-5631.
Zagorski M, Yang J, Shao H, Ma K, Zeng H, Hong A. Methodological and chemical factors affecting amyloid beta peptide amyloidogenicity. Methods Enzymol 1999; 309:189-204.

We claim:

1. A method for treating a peptide and protein folding disorder in a mammalian subject, the method comprising administering to the mammalian subject an effective amount of a composition comprising sterically stabilized simple micelles of a hydrophilic polymer-conjugated lipid or sterically stabilized mixed micelles of a hydrophilic polymer-conjugated lipid and a water-insoluble lipid.

2. The method of claim 1, wherein mammalian subject is a human subject.

3. The method of claim 1, wherein the hydrophilic polymer-conjugated lipid is a phospholipid.

4. The method of claim 1, wherein the hydrophilic polymer is polyethylene glycol (PEG) having a molecular weight of from about 1000 to about 5000.

5. The method of claim 3, wherein the phospholipid is distearoyl phosphatidylethanolamine.

6. The method of claim 1, wherein the water-insoluble lipid is phosphatidylcholine.

7. The method of claim 1, wherein the hydrophilic-polymer-conjugated lipid is distearoyl phosphatidylethanolamine polyethylene glycol 2000 (DSPE-PEG₂₀₀₀).

8. The method of claim 1, wherein the peptide and protein folding disorder being treated is a neurodegenerative disease.

9. The method of claim 8, wherein the neurodegenerative disease is Alzheimer's disease.

10. The method of claim 1, wherein the peptide and protein folding disorder is selected from the group consisting of: alpha-1 antitrypsin deficiency, cystic fibrosis, diabetes type II, hemolytic anemia, Alzheimer's disease for claims because we have data in examples, transmissible spongiform encephalopathies, serpin-deficiency disorders, Huntington disease, Amyotrophic Lateral Sclerosis, Parkinson disease, spinocerebellar ataxias, dialysis-related amyloidosis, polyglutamine diseases, Down's syndrome, Fabry, other gangliosidosis and cataract.

11. The method of claim 1 wherein the composition further comprises a biologically active compound associated with the micelles.

12. The method of claim 11 wherein the biologically active compound is an amphipathic peptide selected from the group consisting of: vasoactive intestinal peptide (VIP), growth hormone releasing factor (GRF), peptide histidine isoleucine (PHI), peptide histidine methionine (PHM), pituitary adenylate cyclase activating peptide (PACAP), gastric inhibitory hormone (GIP), hemodermin, the growth hormone releasing hormone (GHRH), sauvagine and urotensin I, secretin, glucagon, galanin, endothelin, calcitonin, α₁-proteinase inhibitor, angiotensin II, corticotropin releasing factor, antibacterial peptides and proteins in general, surfactant peptides and proteins, α-MSH, adrenolmedullin, ANF, IGF-1, α2 amylin, orphanin, and orexin.

13. The method of claim 1, wherein the composition is delivered intranasally, intravenously, intra-ventrcularly, intracisternally, subcutaneously, topically, intra-thecally, rectally, vaginally, trans-cutaneously, inhalation, sub-lingually, intra-ocular, ocular or orally.

14. A method for treating Alzheimer's Disease (AD) in a mammalian subject by administering to the mammalian subject an effective amount of a composition comprising sterically stabilized simple micelles of a hydrophilic polymer-conjugated lipid or sterically stabilized mixed micelles of a hydrophilic polymer-conjugated lipid and a water-insoluble lipid.

15. The method of claim 14, wherein the mammalian subject is a human subject.

16. The method of claim 14, wherein the hydrophilic polymer-conjugated lipid is distearoyl phosphatidylethanolamine polyethylene glycol 2000 (DSPE-PEG₂₀₀₀).

17. The method of claim 14, wherein the water-insoluble lipid is phosphatidylcholine.

18. The method of claim 14, wherein the composition further comprises a biologically active compound associated with the micelles suitable for treating Alzheimer's Disease.

19. The method of claim 18, wherein the biologically active compound is a member of glucagon/secretin family of peptides.

20. The method of claim 19, wherein the glucagon/secretin family of peptides is selected from the group consisting of vasoactive intestinal peptide (VIP) and pituitary adenylate cyclase activating peptide (PACAP) wherein the PACAP is a L-isomer or D-isomer.

21. The method of claim 14, wherein the composition is delivered intranasally.

22. A method for treating Alzheimer's Disease (AD) in a mammalian subject by administering an effective amount of a composition comprising of a biologically active compound of a member of glucagon/secretin family of peptides associated with sterically stabilized simple micelles of a hydrophilic polymer-conjugated lipid or sterically stabilized mixed micelles of a hydrophilic polymer-conjugated lipid and a water-insoluble lipid.

23. The method of claim 22, wherein the glucagon/secretin family of peptides is selected from the group consisting of: vasoactive intestinal peptide (VIP) and pituitary adenylate cyclase activating peptide (PACAP), wherein the PACAP is a L-isomer or D-isomer.

24. The method of claim 22, wherein the mammalian subject is a human subject.

25. The method of claim 22, wherein the hydrophilic polymer-conjugated lipid is distearoyl phosphatidylethanolamine polyethylene glycol 2000 (DSPE-PEG₂₀₀₀).

26. The method of claim 22, wherein the water-insoluble lipid is phosphatidylcholine.

27. The method of claim 22, wherein the composition is delivered intranasally.
